(19) United States
(12) Patent Application Publication
(10) Pub. No.: US 2025/0050104 A1
(43) Pub. Date: Feb. 13, 2025

(54) ENHANCING NEUROMODULATION FOR SLEEP

(71) Applicant: Boston Scientific Neuromodulation Corporation, Valencia, CA (US)

(72) Inventors: Mahsa Malekmohammadi, Sherman Oaks, CA (US); Hemant Bokil, Cambridge, MA (US); G. Karl Steinke, Valencia, CA (US)

(73) Assignee: Boston Scientific Neuromodulation Corporation, Valencia, CA (US)

(21) Appl. No.: 18/791,850

(22) Filed: Aug. 1, 2024

Related U.S. Application Data
(60) Provisional application No. 63/531,881, filed on Aug. 10, 2023.

(51) Int. Cl. A61N 1/36

(52) U.S. Cl. CPC A61N 1/36078; A61N 1/36132; A61N 1/36139

(57) ABSTRACT

An example method of operating a neuromodulation device to modify or achieve a desired arousal state in a patient may include determining the patient's desired arousal state, detecting one or more signals that are related to the desired arousal state, where the one or more signals are sensed from the patient or provided by an external source. The method also includes determining, via a controller circuit, a therapy parameter setting for the neuromodulation device based on the one or more signals, and generating, via the controller circuit, a control signal in the neuromodulation device to initiate or adjust an arousal state therapy program in accordance with the therapy parameter setting.

### CROSS-REFERENCE TO RELATED APPLICATIONS

This application claims the benefit of U.S. Provisional Patent Application Ser. No. 63/531,881, filed Aug. 10, 2023, which is incorporated herein by reference.

### TECHNICAL FIELD

The disclosure pertains to implantable electrical stimulation systems, and methods for using such systems.

### BACKGROUND

Implantable electrical stimulation systems have proven therapeutic in a variety of diseases and disorders. For example, neuromodulation therapy such as deep brain stimulation (DBS) can be used to treat a variety of neurological diseases and disorders including Parkinson's disease mainly to treat motor symptoms. Sleep disorders such as sleep fragmentation, insomnia and daytime sleepiness often occur in patients with Parkinson's disease and other disorders treated by DBS.

Sleep disturbances and disorders are a known facet of many neurological disorders and it has been proposed that such disturbances may be worsened by conventional DBS. These disturbances are in turn known to be implicated in a wide variety of poor health outcomes. Having a stimulation paradigm that promotes normal sleep/wake cycles could provide greatly improved quality of life to patients. There is an ongoing need to provide alternative medical systems as well as alternative methods for addressing sleep disorders, particularly in patients with Parkinson's disease.

### SUMMARY

This disclosure provides design, material, manufacturing method, and use alternatives for medical devices. A problem to be solved is that of addressing sleep disorders using implantable deep brain stimulation systems.

A first illustrative and non-limiting example takes the form of a neuromodulation system comprising an implantable pulse generator and a lead, the lead comprising a plurality of electrodes adapted for sensing signals in the brain of a patient, the implantable pulse generator comprising a controller circuit adapted to execute a method of modifying or achieving a desired arousal state in a patient, the method comprising: determining the patient's current desired arousal state; detecting one or more signals that are related to the desired arousal state, the one or more signals being sensed from the patient or provided by an external source; determining, via the controller circuit, a therapy parameter setting for the neuromodulation device based on the one or more signals; generating, via the controller circuit, a control signal in the neuromodulation device to initiate or adjust a promoted arousal state therapy program in accordance with the therapy parameter setting; and issuing one or more electrical stimuli via the lead to the patient.

Additionally or alternatively, the controller circuit is configured such that the arousal state is related to sleep, the arousal state therapy program is a sleep therapy program, and determining the patient's desired arousal state includes determining the patient's desired sleep state. Additionally or alternatively, the one or more signals are related to REM sleep behavior disorder (RBD), insomnia, sleep fragmentation, or excessive daytime sleepiness. Additionally or alternatively, the controller circuit is configured to perform detecting one or more signals by detecting one or more signals that are contrary to the desired arousal state.

Additionally or alternatively, when the patient's desired sleep state is a state of wakefulness and the one or more signals relate to excessive daytime sleepiness, the control signal generated via the controller circuit will adjust or switch the neuromodulation device to a program providing stimulation to alleviate or avoid daytime sleepiness. Additionally or alternatively, when the patient's desired sleep state is a state of nighttime sleep and the one or more signals relate to insomnia or sleep fragmentation, the control signal generated via the controller circuit will adjust to or initiate the sleep therapy program of the neuromodulation device to adjust a level of stimulation to promote sleep.

Additionally or alternatively, the controller circuit is configured for determining the patient's desired sleep state by tracking the patient's wakeup time and generating the control signal includes providing a gradual change in stimulation beginning before the patient's wakeup time and reaching wake promoting stimulation at a desired time preceding or aligning with the patient's wakeup time. Additionally or alternatively, the controller circuit is configured for determining the patient's desired sleep state by inputting or tracking the patient's sleep time and generating the control signal includes providing a gradual change in stimulation beginning before the patient's sleep time and reaching a sleep promoting stimulation setting at the patient's sleep time.

Additionally or alternatively, the controller circuit is configured to perform detecting one or more signals by detecting one or more signals that are aligned with the patient's current desired arousal state or with an upcoming arousal state. Additionally or alternatively, the controller circuit is configured to use sensing means to sense the one or more signals from the brain of the patient using the electrodes of the lead. Additionally or alternatively, the controller circuit is configured to obtain the one or more signals from the external source and the external source is one of: sensors or electrodes positioned subcutaneously in the patient and configured to sense physiological data; sensors or electrodes positioned on the skin of the patient and configured to sense physiological data; or a motion sensor worn on the patient and configured to sense kinematic data.

Additionally or alternatively, the controller circuit is configured to obtain the one or more signals from the external source, and the external source is adapted to sense eye movement, eye tracking, drooping eyes, or decreased reaction time. Additionally or alternatively, the controller circuit is configured to obtain the one or more signals from the external source, and the external source is a cellphone or computer application configured to sense signals from the patient or obtain patient feedback. Additionally or alternatively, wherein the one or more signals include data of a calendar or schedule. Additionally or alternatively, after generating the control signal to initiate or adjust the arousal state therapy program, the controller circuit is further configured to wait a pre-determined period of time and re-evaluate the one or more signals and/or request the patient's input regarding effectiveness of the arousal state therapy program.

Another illustrative and non-limiting example takes the form of a method of operating a neuromodulation device to modify or achieve a desired arousal state in a patient, the method comprising: determining the patient's current desired arousal state; detecting one or more signals that are related to the desired arousal state, the one or more signals being sensed from the patient or provided by an external source; determining, via a controller circuit, a therapy parameter setting for the neuromodulation device based on the one or more signals; and generating, via the controller circuit, a control signal in the neuromodulation device to initiate or adjust a promoted arousal state therapy program in accordance with the therapy parameter setting.

Additionally or alternatively, the arousal state is related to sleep, the arousal state therapy program is a sleep therapy program, and determining the patient's desired arousal state includes determining the patient's desired sleep state. Additionally or alternatively, the one or more signals are related to REM sleep behavior disorder (RBD), insomnia, sleep fragmentation, or excessive daytime sleepiness. Additionally or alternatively, when the patient's desired sleep state is a state of nighttime sleep and the one or more signals relate to insomnia or sleep fragmentation, the control signal generated via the controller circuit will adjust to or initiate the sleep therapy program of the neuromodulation device to adjust a level of stimulation to promote sleep.

Additionally or alternatively, determining the patient's desired sleep state includes tracking the patient's wakeup time and generating the control signal includes providing a gradual change in stimulation beginning before the patient's wakeup time and reaching wake promoting stimulation at a desired time preceding or aligning with the patient's wakeup time. Additionally or alternatively, determining the patient's desired sleep state includes inputting or tracking the patient's sleep time and generating the control signal includes providing a gradual change in stimulation beginning before the patient's sleep time and reaching a sleep promoting stimulation setting at the patient's sleep time.

Additionally or alternatively, detecting one or more signals includes detecting one or more signals that are contrary to the desired arousal state. Additionally or alternatively, detecting one or more signals includes detecting one or more signals that are aligned with the patient's current desired arousal state or with an upcoming arousal state. Additionally or alternatively, the one or more signals are sensed from the patient and include physiological data from sensors or electrodes positioned subcutaneously or on the patient's skin. Additionally or alternatively, the one or more signals are sensed from the patient and include kinematic data from a worn motion sensor. Additionally or alternatively, the one or more signals are sensed from the patient and include behavioral tracking data including eye tracking, drooping eyes, or decreased reaction time. Additionally or alternatively, the one or more signals are sensed from the patient and include patient feedback received through a cellphone or computer application. Additionally or alternatively, the one or more signals are provided by the external source and include a clock, calendar, or schedule.

Additionally or alternatively, when the patient's desired sleep state is a state of wakefulness and the one or more signals relate to excessive daytime sleepiness, the control signal generated via the controller circuit will adjust or switch the neuromodulation device to a program providing stimulation to alleviate or avoid daytime sleepiness. Additionally or alternatively, the one or more signals are sensed from the patient and include signals indicative of patient brain activity. Additionally or alternatively, after generating the control signal to initiate or adjust the arousal state therapy program, the method further comprises waiting a pre-determined period of time and re-evaluating the one or more signals and/or requesting the patient's input regarding effectiveness of the arousal state therapy program.

Another illustrative and non-limiting example takes the form of a method of operating a neuromodulation device to promote sleep or wakefulness in a patient, the method comprising: determining a desired sleep state from a schedule of designated sleep hours; detecting one or more signals related to insomnia, sleep fragmentation, or excessive daytime sleepiness, the one or more signals being sensed from the patient or provided by an external source; determining, via a controller circuit, a therapy parameter setting for the neuromodulation device based on the one or more signals, the therapy parameter setting including a stimulation energy; and generating, via the controller circuit, a control signal in the neuromodulation device to initiate or adjust a sleep therapy program and apply the stimulation energy in accordance with the therapy parameter setting.

Additionally or alternatively, when the desired sleep state is a state of wakefulness and the one or more signals relate to excessive daytime sleepiness, the control signal generated via the controller circuit will switch the neuromodulation device to a program providing stimulation to alleviate or avoid daytime sleepiness. Additionally or alternatively, when the desired sleep state is a state of nighttime sleep and the one or more signals relate to insomnia or sleep fragmentation, the control signal generated via the controller circuit will initiate the sleep therapy program of the neuromodulation device to adjust a level of stimulation to promote sleep.

Another illustrative and non-limiting example takes the form of a method of operating a neuromodulation device to promote sleep or wakefulness in a patient, the method comprising: determining a desired sleep state; detecting one or more signals related to insomnia, sleep fragmentation, or excessive daytime sleepiness, the one or more signals being derived from the patient's physiological data or provided by an external source; determining, via a controller circuit, a therapy parameter setting for the neuromodulation device based on the one or more signals; and generating, via the controller circuit, a control signal in the neuromodulation device to initiate or adjust a sleep therapy program in accordance with the therapy parameter setting; wherein when the desired sleep state is a state of wakefulness and the one or more signals relate to excessive daytime sleepiness, the control signal generated via the controller circuit will switch the neuromodulation device to a program providing stimulation to alleviate or avoid daytime sleepiness; wherein when the desired sleep state is a state of nighttime sleep and the one or more signals relate to insomnia or sleep fragmentation, the control signal generated via the controller circuit will initiate the sleep therapy program of the neuromodulation device to adjust a level of stimulation to promote sleep.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a flow chart illustrating a method of operating a neuromodulation device to promote sleep or wakefulness in a patient; and

FIG. 2 illustrates an implantable deep brain stimulation system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular method step, feature, structure, or characteristic, but every embodiment may not necessarily include the particular method step, feature, structure, or characteristic. Moreover, such phrases are not necessarily referring to the same embodiment. Further, when a method step, feature, structure, or characteristic is described in connection with an embodiment, it would be within the knowledge of one skilled in the art to affect the particular method step, feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

The following description should be read with reference to the drawings, which are not necessarily to scale, wherein similar elements in different drawings are numbered the same. The description and drawings are intended to illustrate but not limit the disclosure. Those skilled in the art will recognize that the elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Sleep disorders such as REM sleep behavior disorder (RBD), sleep fragmentation, insomnia and daytime sleepiness often occur in patients with Parkinson's disease and other neurological disorders that may be treated by deep brain stimulation (DBS). Dominant symptoms of such neurological disorders are often most successfully treated by DBS. For example, in the case of Parkinson's disease and other movement disorders, motor symptoms are successfully treated by DBS. However, DBS remains inadequate in directly addressing sleep disorders in these patients. Sleep disorders, such as falling asleep unintentionally while conducting daily activities, could result in harm to the patient and others. Additionally, the lack of adequate and/or restful sleep may reduce a patient's quality of life and well-being, and may affect other aspects of their life such as response to medications and/or therapies, and may accelerate disease progression.

Some of the target areas in DBS for treatment of movement disorders such as the thalamus, subthalamic nucleus (STN), and globus pallidus internus (GPi), in addition to their critical involvement in the motor circuitry, are also parts of the arousal system, and their activities are modulated by various states of sleep and arousal. Other targets from the arousal circuitry of the brain have been investigated for DBS to improve sleep disorders, e.g. Pedunculopontine nucleus (PPN) and external globus pallidus (GPe).

Electrophysiological activities recorded from these and other areas, via sensing elements in the DBS systems are helpful in identifying sleep and awake states. These signals and/or other related physiological and surrogate signals may be used to identify the emergence of an episode of daytime sleepiness and guide change of stimulation, including to secondary programs that may result in restoration of arousal. Examples of physiological and surrogate signals may include any signal indicating the time of day or circadian rhythms, such as eye tracking, heart rate, respiratory rate, and motion detection by a body worn accelerometry sensor, signals based on access to or interaction with electronic systems, such as computers, phones, apps, cloud-based software, and other zeitgebers, including chronobiological and other. Tracking of physiological and behavioral markers during sleep may also be used to guide change to stimulation to optimize sleep quality.

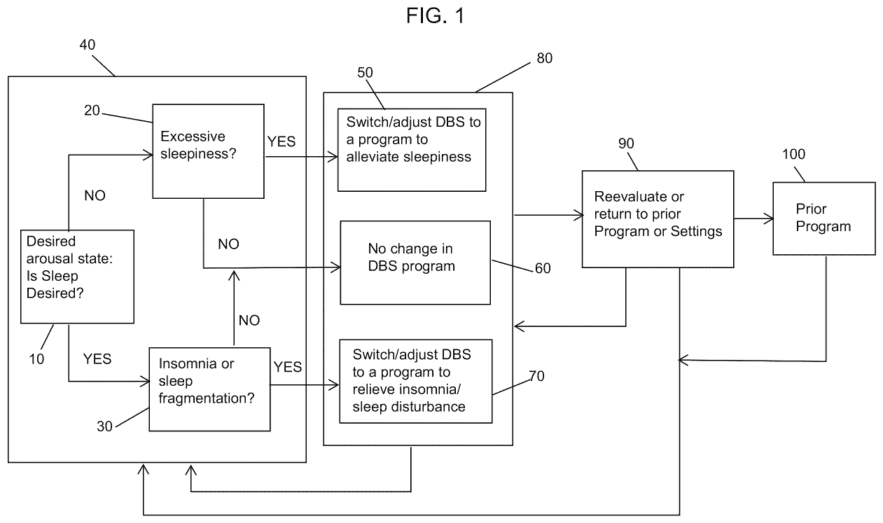

FIG. 1 is a flow chart illustrating a method of operating a neuromodulation device to modify or achieve a desired arousal state in a patient. In some examples, the arousal state may be related to sleep. The method may promote sleep or wakefulness in the patient. The method may involve changing when, where and how to stimulate the patient's brain to address sleep problems, and also initiate new stimulation areas that can help treat sleep disorders. The method may identify when sleep problems occur and utilize a variety of sensors to track issues and then use the electrodes of a DBS system to adjust brain stimulation to help the patient sleep or remain awake as desired. The patient or their care giver alternatively could manually activate the system when a change in the arousal state is noticed and the system may adjust stimulation accordingly. Once a patient has a DBS system implanted and adjusted for movement control, the patient may utilize the system to address any sleep issues they may have.

Turning to the figure, step 10 involves determining the patient's desired arousal state, such as whether sleep is desired or not. This may be achieved by a direct query asking if the patient wishes to be asleep or awake. The query may be posed through the DBS remote control or through a cellphone or computer application. One element that may be considered in determining the patient's desired sleep state in step 10 may be comparing the current time of day and with a schedule input by the patient indicating daily times of desired sleep and wakefulness. In this way, the desired arousal state takes into consideration “appropriate expectations”; i.e., sleeping during night, being awake during daytime. Alternatively, the controller circuit may learn the patient's schedule by tracking sleep and awake times. However, the schedule may be overridden by direct user input, for example in a situation where the patient desires to stay awake during a time period the schedule indicates would ordinarily be for sleeping. This may be of particular use when the patient is traveling across time zones or desires their system to adapt to any non-standard sleep events. The method may be triggered or activated automatically by an event such as sensors determining the patient is nodding off during a time period scheduled for wakefulness or activity levels indicative of wakefulness during a time period scheduled for sleep. Alternatively, the patient or caregiver may manually activate the method.

If step 10 results in a determination that sleep is not desired, step 20 involves determining whether the patient is excessively sleepy. If not, no change is made to the neuromodulation program at step 60. If sleep is desired, step 30 involves determining whether the patient is experiencing insomnia or sleep fragmentation. If not, no change is made to the neuromodulation program at step 60. Both steps 20 and 30 may include evaluating direct input from the patient and/or detecting one or more signals that are related to the desired arousal, or sleep state. Additionally, signals related to an undesired arousal or sleep state may be detected. These signals may be sensed from the patient or may be provided by an external source. The signals may be contrary to the desired sleep state, indicating that a change or modification to the stimulation provided by the DBS may be needed. In other examples, the signals may help anticipate an upcoming desired arousal state. For example, when signals indicate the patient will soon desire a state of sleep, the level and/or location of stimulation may be altered to match the desired sleep state. Steps 10, 20, and 30 together make up a Detection Unit 40.

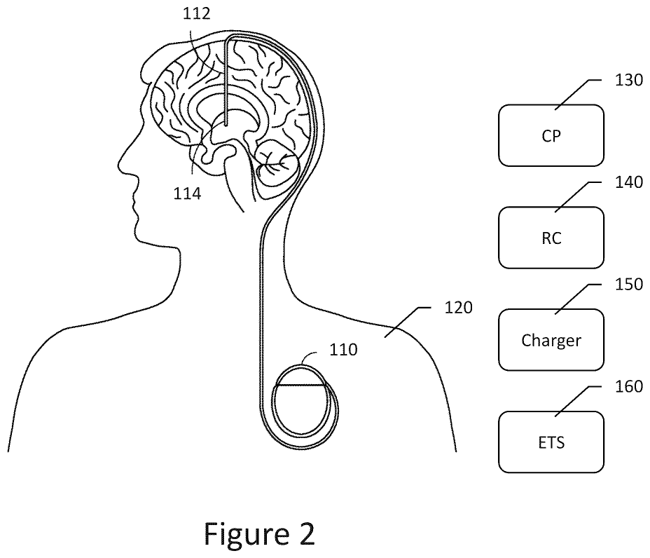

Patient inputs may be received via a patient remote control (RC 140; see FIG. 2), which may be implemented as the patient's own smartphone or as a dedicated device. Additionally, the patient inputs may be provided by a care giver. The patient may provide an active input as desired by the patient, or in response to prompts and/or queries by the RC, or passively by the RC sensing activity (such as gait, tremor, etc.), or timing of usage of other devices, such as a smartphone (for example, insomnia may be indicated by the use of a smartphone late at night, or use of the RC to adjust therapy at a time when sleep is desired/expected). Sensing may also occur using the electrodes and/or circuitry of an implanted DBS system. For example, as discussed relative to FIG. 2, below, a plurality of electrodes may be positioned in proximity to target tissue for therapy purposes. Those same electrodes can be used to sense the physiological signals discussed above, for example by sensing for particular signal features such as signal peaks, signal shapes, signal frequencies, signal amplitudes or other parameters of the sensed signals. In some examples, sensing may be performed using electrodes that are not selected as active therapy electrodes, and/or at times that do not correspond to therapy being output.

If step 10 indicates the desired sleep state is a state of wakefulness, and the determination in step 20 is that the patient is experiencing excessive sleepiness, then the method includes step 50 which involves determining, via a controller circuit, a therapy parameter setting for the neuromodulation device based on the one or more signals that are contrary to the desired sleep state, and then switching the neuromodulation device, such as a DBS system, to a stored program that is designed to alleviate or avoid sleepiness, or adjusting the current program to better alleviate or avoid sleepiness. In some examples, adjusting the DBS in step 50 may include a calibration step in which the stored program may be calibrated by the patient to achieve the desired result of alleviating sleepiness in the patient.

Changing or adjusting the program may involve generating, via the controller circuit, a control signal in the neuromodulation device to initiate or adjust a sleep therapy program in accordance with the therapy parameter setting. The therapy program may involve providing electrical stimulation to various regions of the patient's brain through electrodes in the DBS system. For example, if the current program is one designed for or known to promote sleep, the DBS system may switch to a different program with increased stimulation or stimulation in different areas that promote wakefulness. If the current program is one designed for wakefulness, the program may be adjusted to provide one or more of an increase in level of stimulation, an increase in time of stimulation, or target different areas for stimulation. Any adjustable DBS parameter may be changed in order to adjust the program to provide the desired level of sleep promotion or wakefulness. Rate, amplitude, pulse width, pulse shape, location of stimulation (current fractionalization), as well as the addition of stimulation in other implanted leads if present, and/or interplay of multiple leads. In some embodiments, step 50 may first involve the controller circuit assessing multiple programs for listed efficacy and/or side effects regarding sleep. For example, the DBS program may be switched to one particularly optimized to avoid daytime sleepiness. As any alterations are made to a program, the alterations may be annotated and saved to provide information for future assessments. Such changes may be implemented automatically in an implanted pulse generator, or may occur as prompted by a patient RC 140, for example.

Similarly, if step 10 indicates the desired state is being asleep and the determination in step 30 is that the patient is experiencing insomnia or sleep fragmentation, then the method includes step 70 which involves determining, via a controller circuit, a therapy parameter setting for the neuromodulation device based on the one or more signals that are contrary to the desired sleep state, and then switching the neuromodulation device, such as a DBS system, to a stored program that is designed to relieve insomnia and sleep disturbance and encourage deeper and/or more continuous sleep, or adjusting the current program to better encourage sleep. Changing or adjusting the program may include merging elements of one program with elements of another program. Changing or adjusting the program may involve generating, via the controller circuit, a control signal in the neuromodulation device to initiate or adjust a sleep therapy program in accordance with the therapy parameter setting. For example, the DBS may switch from the current first program to a second program with decreased stimulation or stimulation in areas that promote sleep. Alternatively, if the current program is one designed for sleep, the program may be adjusted to decrease the level of stimulation or target different areas for stimulation.

In addition to steps 60 and 70 determining a therapy parameter setting and then generating a control signal to initiate or adjust the therapy program to maintain a current sleep state or change the sleep state, the control circuit may be configured to track the patient's wakeup time and then the step of generating the control signal includes providing a gradual change (such as an increase) in stimulation or change in other parameters of stimulation beginning before the patient's wakeup time and reaching wake promoting stimulation at a desired time preceding or aligning with the patient's wakeup time. Similarly, the control circuit may track the patient's sleep time such that the step of generating the control signal includes providing a gradual change (such as a decrease) in stimulation or change in other parameters of stimulation beginning before the patient's sleep time and reaching a sleep promoting stimulation setting at the patient's sleep time. The sleep promoting stimulating setting may be the lowest level of stimulation.

Steps 50, 60, and 70 together make up a learning and decision unit 80. After switching to a new program or adjusting the current program of the neuromodulation device in step 50 or 70, the method may include the step 90 of determining when to reevaluate the patient's sleep state by returning to the Detection Unit 40 and starting over with step 10, return to the learning and decision unit 80 and switch or adjust the program, or return to a prior program or settings in step 100.

The Detection Unit 40 may use one or multiple arrays of input data and a processing unit that will identify the underlying arousal state of the patient from the input data. Such input data may include, for example data sensed by an implantable pulse generator, data sensed by an RC, data entered by a patient in the RC, or data obtained from additional devices such as personal devices of the patient.

In steps 20 and 30, detecting one or more signals that are contrary to the desired sleep state may involve input data and signals sensed from the patient. The input data may include any or a combination of physiological data, kinematic data, other types of behavioral tracking, patient feedback, and other outside sources. Signals may include input provided directly by the patient such as entering into an input device an indication the patient is sleepy and wants to stay awake, wants to sleep now but is wide awake, wants to sleep now keeps waking up, or other similar message. The input from the patient may be provided in response to an inquiry from the neuromodulation device or the patient may selectively provide information. The input may be entered into a remote-control device linked to the DBS system.

In addition to direct patient input, the signals may be sensed from the patient's body and include signals indicative of patient brain activity received by the neuromodulation device. For example, brain activity indicative of wakefulness during the patient's scheduled sleep time in step 30 or brain activity indicative of sleep during the patient's scheduled awake time in step 20 may be used by the controller circuit to determine a therapy parameter setting for the DBS system. Physiological data may be directly recorded from the DBS lead, other electrodes in the brain, such as in the cortical region, or may be in the form of evoked or spontaneous signals, using time or frequency domain signals or derivatives. Physiological data may also be recorded from outside the DBS system, such as wearable EEG sensors linked to a cellphone application. Signals indicative of patient brain activity may include electrical, magnetic, blood-flow, glucose processing, etc. In other examples, sensors or electrodes worn on or in the body, including those positioned subcutaneously, on the skin surface, or positioned adjacent the body may provide additional physiological data.

In steps 20 and 30, signals may include physiological data from sensors or electrodes positioned subcutaneously or on the patient's skin. Such signals may include one or more of electrocardiogram (ECG), heart rhythm, breathing rhythm, body temperature, skin conductivity, electromyogram (EMG) from muscles to track movements, and blood flow either peripherally or in the brain. These signals may also be indirectly recorded using sleep tracking devices such as a smart watch or smart rings, etc. Both excessive sleepiness and insomnia or sleep fragmentation may be determined by comparing current physiologic data of one or more of the above parameters to a previous recording of physiologic data for the same parameters when the patient was asleep or awake.

The one or more signals contrary to the desired sleep state measured in steps 20 and 30 may also be provided by an external source. For example, signals may be sensed from devices worn by the patient such as kinematic data from a motion sensor. Additional signals may include behavioral tracking data including tracking involuntary eye movements (saccades), drooping of the eyes, or decreased reaction time. These signals may be received from cameras or other external sensors in the patient's environment.

Patient feedback may also be used as the one or more signals in steps 20 and 30. the patient feedback may be provided through interaction with a cellphone or computer application, the remote control associated with the DBS system, or other external sources. For example, when the patient is using a computer or cellphone, and application on the device may recognize a delay in the patient's reaction time or attention and pose a query asking if the patient would like assistance in staying awake. Further external sources that may provide signals include a clock, calendar, or schedule, which may be in communication with the controller circuit such as via Wi-Fi or Bluetooth connection.

The Learning and Decision Unit 80 performs steps 50 or 70, depending on process flow, resulting in a change and/or alteration of the stimulation program of the neuromodulation device that achieves the desired sleep state for the patient. The learning and decision unit 80 may keep a history of one or more of these programs or subunits such as Area (indicating which electrodes are sending signals), or super units such as the Program Schedule, that have been optimized for one or more of the desired sleep states. The Learning and Decision unit 80 can “learn” to adjust the best setting as new data from the same patient, or a group of patients become available to it. Learning groups can be localized based on multiple needs. The Learning and Decision Unit 80 may operate solely on a single patient, be restricted to data from a single patient, and be located within a single patient's constellation of devices. In other embodiments, the Learning and Decision Unit 80 may comprise cloud and/or connected components, operating on a single patient at a time, with or without additional knowledge of aggregates, where the aggregates are able to be formed at a practice, practice group, region, country, or on a global level.

The Learning and Decision Unit 80 may keep track of time of day and start adjusting stimulation to achieve optimal sleep quality and optimal alertness. For example, a sleep program may start at 8 pm and gradually adjust stimulation to achieve an optimal state of sleepiness at 10 pm. Similarly, a wakefulness program may start at 6 am and gradually adjust stimulation to achieve optimal wakefulness at 7 am. These adjustments will ideally be imperceptible to patient, especially in terms of their effect on motor or other symptoms and/or side effects.

The Learning and Decision Unit 80 may keep track of and adjust the program and stimulation to seasons, time zones, and other differences noted in the patient's calendar. For example, program alterations may be made for travel, particularly across time zones, and for holidays when the patient may not desire to wake and the usual time. Also, stimulation for winter months may be different from that for the summer months, particularly in northern regions, reflecting differences in sleep need and quality. These external inputs may be provided from the patient's calendar. In all of the above methods, the patient may always override any wakefulness or sleep program by inputting information into the DBS indicating a current desire to sleep or stay awake, regardless of the time of day or schedule.

During steps 50 or 70, the adjustments to stimulation programs may be assisted with Image Guided Programming (IGP) to determine location and timing of stimulation, magnitude, pulse width, and other parameters. Examples of using IGP are discussed in U.S. Pat. Nos. 9,959,940, 10,342,972, 10,350,413, 10,716,946, and 11,602,635, the content of each of which is hereby incorporated herein by reference in its entirety. Using IGP, the control circuitry may determine possible stimulation field model (SFM) adjustments and/or additions and/or subtractions to increase or reduce wakefulness or sleep as desired by the patient and based on the analysis of neuroanatomical data. This may include the creation of additional Programs, addition of stimulation areas, or modification of how stimulation patterns are assessed for utility by the addition or removal of target or avoid structures in the patient's neural structure. For example, some systems may use a method of identifying well suited stimulation program parameters for a patient by identifying target structures (those regions associated with clinical benefit) and avoid structures (those regions associated with side effects or likely side effects), and overlapping the SFM with such target and avoid regions to yield a metric indicating how well the particular parameters associated with the SFM will do at achieving beneficial stimulation while limiting side effects. Some examples for IGP can be found in U.S. Pat. No. 11,195,609, the disclosure of which is incorporated herein by reference.

Adjustments coming from IGP can then be pushed out to the DBS system. Such adjustment may be with or without the user's knowledge—that is by pushing through the RC to obtain the patient approval, or by simply loading to the implantable pulse generator, or by creating the program updates within the pulse generator itself. Adjustment of therapy parameters/stimulation settings can include, for example, including increasing or decreasing amplitude, changing pulse width or pulse repetition rate (frequency), or modifying the electrode utilization to steer the stimulation field such as by rotation, movement up or down, expansion, contraction, etc.

Additionally, using IGP, the control circuitry may determine possible program changes and/or additions. For example, a motor program (minimizing tremor or allowing greater control over movement) may run for a predetermined period of time (most of the day, for example), but in the evening a supplemental “Sleep Time” program can be initiated to reduce, mitigate or offset side effects induced by the motor program, to allow the user to sleep. In another example, a “Sleep” program is set to run and at some time before the wake-up time, a “Wake Up” program is started. The program amplitudes may be ramped up gradually, for example, over a time frame of one minute to one hundred minutes, or for about an hour, for example, to achieve the optimal sleepiness stage or the optimal wakefulness state. These programs may include patterns including, without limitation, burst patterns (groups of pulses delivered with a relatively shorter intra-burst period and a relatively longer inter-burst period, as known in the art), and they may also target specific neuronal elements to achieve the desired goals of sleep or wakefulness.

An IGP method may also be used to identify optimized stimulation programs, patterns, or SFM shapes for different purposes for one patient to use throughout the day. For example, using a first selection of target and avoid structures tailored for motor control, a first optimized therapy can be identified. A second selection of target and avoid structures, tailored for relaxation, may be used to identify a second optimized therapy. A third selection of target and avoid structures tailored for sleep may be used to identify a third optimized therapy, to use when the patient is going to sleep. A fourth selection of target and avoid structures can be used to identify a fourth optimized therapy to be used in the pre-wake-up time period. Additional modifications to existing IGP therapy may be used during various stages of sleep (REM vs core vs deep sleep) as well.

In other embodiments, the Learning and Decision Unit 80 may be tuned to adjust the stimulation to both target sleep and/or wakefulness, or to do more to avoid side effects of sleep or wakefulness in a state dependent manner. For example, the Learning and Decision Unit 80 may adjust for both optimal sleep and wakefulness, putting stimulation on a sleep sweet spot, and also moving stimulation off a sleep sour spot. Both optimal sleep and wakefulness programs may overlap, and other spatio-temporal parameter optimizations (e.g., SFM same, but pulse repetition rate, frequency, or pulse pattern changes, such as modifying a burst pattern) may be used to achieve a desired level of sleep or wakefulness that satisfies the patient.

After the Learning and Decision Unit 80 switches to a new program or adjusts the current program of the neuromodulation device in step 50 or 70, or makes no change to the program in step 60, the method may include the step 90 of determining when to re-evaluate the patient's sleep state by returning to the Detection Unit 40 and starting over with step 10, or return to a prior program or settings in step 100. The method may include waiting a pre-determined period of time and then re-evaluating the signals that were previously contrary to the desired sleep state. If those signals are now in accordance with the desired sleep state, the new program or adjusted program parameters and annotations regarding what alterations were made may be saved as a program known to achieve a particular sleep state. The saved program may include all data used in the method, including the initial desired sleep state, signals that were contrary to the desired sleep state, selected new program or altered therapy parameters and control signals, and level, location, and duration of stimulating therapy applied to achieve the desired sleep state. When the desired sleep state is wakefulness, the method may also include requesting patient input regarding the effectiveness of the sleep therapy program. The step of re-evaluating the signals and/or requesting patient feedback, or the entire method may be repeated at various times during a scheduled sleep state. In other embodiments, the entire method may be repeated a set number of times throughout each 24-hour day, providing feedback for both sleep states and wakefulness states.

Programs optimized for sleep and/or wake cycles may conflict with those optimized to control symptoms. In that case state dependent prioritization may help select the most appropriate program or the patient or their caregiver may be able to directly override the automatic program selection. This may present a minor challenge in that it may take time to find or create a program that is optimal for all aspects of the patient's life. For example, the optimal program for sleep and wakefulness may conflict with symptom management, and the optimal sleep program may allow more tremors. The method may be adjusted and the patient will determine which things are more important to reduce conflicts. In particular, there may be times when the patient values sleep over tremor control, and vice versa. Patient input into the DBS system may always override the program and provide the learning and program adjustment needed to achieve a balance that is satisfactory to the patient.

In some examples, the various changes made to optimize sleep and/or wake cycles, as well as the effect such changes have on the desired therapy program(s) and the results achieved by therapy programs, are recorded and may be used as a further input to further develop data about the patient's brain. For example, if program intended to induce sleep is observed to result in additional tremors, the target/avoid structure map for the patient, used in to develop the IGP, can be augmented with this additional data. Doing so will allow the trial and error that may be associated with efforts to enhance sleep/awake phases for the patient to be directed toward a beneficial outcome.

FIG. 2 shows an illustrative DBS system implanted in a patient. The system comprises a pulse generator 110, shown implanted in the pectoral region of a patient 120. The pulse generator 110 is coupled to a lead 112 which extend subcutaneously to the head of the patient 120, through a burr hole formed in the patient's skull, and then into the brain of the patient. In the example shown, the lead 112 includes a plurality of electrodes positioned near the distal end 114 of the lead. The lead 112 may be placed at any suitable location of the brain where a target for therapy is identified. For example, a lead 112 may be positioned so that the distal end 114 is near the mid-brain and/or various structures therein, such as the sub-thalamic nucleus, that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulata, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice.

Conditions to be treated may include dementia, Alzheimer's disease, Parkinson's disease, various tremors, depression, anxiety or other mood disorders, sleep related conditions, etc. Therapeutic benefits may include, for example, and without limitation, improved cognition, alertness, and/or memory, enhanced mood or sleep, avoidance of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of FIG. 2 includes various external devices. A clinician programmer (CP) 130 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 130 can be used by a physician, or at the direction of a physician, to obtain data from and provide instructions the pulse generator 110 via suitable communications protocols such as Bluetooth or MedRadio or other wireless communications protocols, and/or via other modalities such as inductive telemetry.

A patient remote control (RC) 140 can be used by the patient to perform various actions relative to the pulse generator 110. These may be physician defined options, and may include, for example, turning therapy on and/or off, entering requested information (such as answer questions about activities and therapy benefits and side effects), and making (limited) adjustments to therapy such as selecting from available therapy programs and adjusting, for example, amplitude settings. The RC 140 can communicate via similar telemetry as the CP 130 to control and/or obtain data from the pulse generator 110. The patient RC 140 may also be programmable on its own, or may communicate or be linked with the CP 130.

A charger 150 may be provided to the patient to allow the patient to recharge the pulse generator 110, if the pulse generator 110 is rechargeable. Some pulse generators 110 are not rechargeable, and so the charger 150 may be omitted. The charger 150 can operate, for example, by generating a varying magnetic field to activate an inductor associated with the pulse generator 110 to provide power to recharge the pulse generator, using known methods.

Some systems may include an external test stimulator (ETS) 160. The ETS 160 can be used to test therapy programs after the lead 112 has been implanted in the patient to determine whether therapy is or can work for the patient 120. For example, an initial implantation of the lead 112 can take place using, for example, a stereotactic guidance system, with the pulse generator 110 temporarily left out. The lead 112 may have a proximal end thereof connected to an intermediate connector (sometimes called an operating room cable) that couples to the ETS 160. After lead 112 has been implanted and coupled to the ETS 160, the ETS can be programmed using the CP 130 with various therapy programs and stimulation parameters. The patient 120 may then test the therapy programs using the RC 140 to select programs to test during a trial period. Once therapy suitability for the patient is established to the satisfaction of the patient 120 and/or physician, the permanent pulse generator 110 is implanted and the lead 112 is connected thereto, with the ETS then being removed from use.

The pulse generator 110 may include separate circuits, sometimes referred to as operational circuitry, including a microcontroller (which may also be implemented as part of a microprocessor if desired), which controls operations of the pulse generator at a high level. Various control circuit implementations are well known in the art, including in commercial products such as the Vercise Genus™ DBS System, and other competing deep brain stimulation systems that are commercially available and well known to the skilled person. The pulse generator 110 can include a power source, typically a battery (rechargeable or primary cell, as desired), though some systems may be adapted to operate without a battery by receiving power inductively or through other link (such as radiofrequency) and issuing therapy using the received power without long-term storage. The microcontroller may include memory for storing operational instructions in a non-transient media, such as a Flash memory, RAM, ROM, etc. Various associated control and sensing circuits may be included, such as one or more application specific integrated circuits (ASICs) for sensing electrical signals obtained from the lead 112 and electrodes thereon, such as amplification, filtering and analog-to-digital conversion circuitry.

The pulse generator 110 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of the pulse generator 110, CP 130, RC 140, Charger 150, and ETS 160 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The lead 112 may be, for example and without limitation, a paddle lead or a linear lead. Some examples may use a directional lead, having one or more segmented electrodes, with or without ring electrodes, allowing the stimulation field to be finely tuned to desired targets using, for example, a plurality of voltage sources or by using a pulse generator having multiple independent current control (MICC). MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes on the lead. It should be noted that the pulse generator canister may serve as an indifferent electrode or as a return electrode for therapy outputs; if desired, one of the electrodes on the lead itself may instead be used as a return electrode. Thus, for example, the electrodes on the lead may serve as cathodes while pulse generator canister serves as an anode during one phase of stimulation pulse delivery.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A method of operating a neuromodulation device to modify or achieve a desired arousal state in a patient, the method comprising:
determining the patient's current desired arousal state;
detecting one or more signals that are related to the desired arousal state, the one or more signals being sensed from the patient or provided by an external source;
determining, via a controller circuit, a therapy parameter setting for the neuromodulation device based on the one or more signals; and
generating, via the controller circuit, a control signal in the neuromodulation device to initiate or adjust a promoted arousal state therapy program in accordance with the therapy parameter setting.

2. The method of claim 1, wherein the arousal state is related to sleep, the arousal state therapy program is a sleep therapy program, and determining the patient's desired arousal state includes determining the patient's desired sleep state.

3. The method of claim 2, wherein the one or more signals are related to REM sleep behavior disorder (RBD), insomnia, sleep fragmentation, or excessive daytime sleepiness.

4. The method of claim 2, wherein when the patient's desired sleep state is a state of nighttime sleep and the one or more signals relate to insomnia or sleep fragmentation, the control signal generated via the controller circuit will adjust to or initiate the sleep therapy program of the neuromodulation device to adjust a level of stimulation to promote sleep.

5. The method of claim 2, wherein determining the patient's desired sleep state includes tracking the patient's wakeup time and generating the control signal includes providing a gradual change in stimulation beginning before the patient's wakeup time and reaching wake promoting stimulation at a desired time preceding or aligning with the patient's wakeup time.

6. The method of claim 2, wherein determining the patient's desired sleep state includes inputting or tracking the patient's sleep time and generating the control signal includes providing a gradual change in stimulation beginning before the patient's sleep time and reaching a sleep promoting stimulation setting at the patient's sleep time.

7. The method of claim 1, wherein detecting one or more signals includes detecting one or more signals that are contrary to the desired arousal state.

8. The method of claim 1, wherein detecting one or more signals includes detecting one or more signals that are aligned with the patient's current desired arousal state or with an upcoming arousal state.

9. The method of claim 1, wherein the one or more signals are sensed from the patient and include physiological data from sensors or electrodes positioned subcutaneously or on the patient's skin.

10. The method of claim 1, wherein the one or more signals are sensed from the patient and include kinematic data from a worn motion sensor.

11. The method of claim 1, wherein the one or more signals are sensed from the patient and include behavioral tracking data including eye tracking, drooping eyes, or decreased reaction time.

12. The method of claim 1, wherein the one or more signals are sensed from the patient and include patient feedback received through a cellphone or computer application.

13. The method of claim 1, wherein the one or more signals are provided by the external source and include a clock, calendar, or schedule.

14. The method of claim 1, wherein when the patient's desired sleep state is a state of wakefulness and the one or more signals relate to excessive daytime sleepiness, the control signal generated via the controller circuit will adjust or switch the neuromodulation device to a program providing stimulation to alleviate or avoid daytime sleepiness.

15. The method of claim 1, wherein the one or more signals are sensed from the patient and include signals indicative of patient brain activity.

16. The method of claim 1, where after generating the control signal to initiate or adjust the arousal state therapy program, the method further comprises waiting a pre-determined period of time and re-evaluating the one or more signals and/or requesting the patient's input regarding effectiveness of the arousal state therapy program.

17. A method of operating a neuromodulation device to promote sleep or wakefulness in a patient, the method comprising:
determining a desired sleep state from a schedule of designated sleep hours;
detecting one or more signals related to insomnia, sleep fragmentation, or excessive daytime sleepiness, the one or more signals being sensed from the patient or provided by an external source;
determining, via a controller circuit, a therapy parameter setting for the neuromodulation device based on the one or more signals, the therapy parameter setting including a stimulation energy; and
generating, via the controller circuit, a control signal in the neuromodulation device to initiate or adjust a sleep therapy program and apply the stimulation energy in accordance with the therapy parameter setting.

18. The method of claim 17, wherein when the desired sleep state is a state of wakefulness and the one or more signals relate to excessive daytime sleepiness, the control signal generated via the controller circuit will switch the neuromodulation device to a program providing stimulation to alleviate or avoid daytime sleepiness.

19. The method of claim 17, wherein when the desired sleep state is a state of nighttime sleep and the one or more signals relate to insomnia or sleep fragmentation, the control signal generated via the controller circuit will initiate the sleep therapy program of the neuromodulation device to adjust a level of stimulation to promote sleep.

20. A method of operating a neuromodulation device to promote sleep or wakefulness in a patient, the method comprising:
determining a desired sleep state;
detecting one or more signals related to insomnia, sleep fragmentation, or excessive daytime sleepiness, the one or more signals being derived from the patient's physiological data or provided by an external source;
determining, via a controller circuit, a therapy parameter setting for the neuromodulation device based on the one or more signals; and
generating, via the controller circuit, a control signal in the neuromodulation device to initiate or adjust a sleep therapy program in accordance with the therapy parameter setting;
wherein when the desired sleep state is a state of wakefulness and the one or more signals relate to excessive daytime sleepiness, the control signal generated via the controller circuit will switch the neuromodulation device to a program providing stimulation to alleviate or avoid daytime sleepiness;
wherein when the desired sleep state is a state of nighttime sleep and the one or more signals relate to insomnia or sleep fragmentation, the control signal generated via the controller circuit will initiate the sleep therapy program of the neuromodulation device to adjust a level of stimulation to promote sleep.
